(19) United States
(12) Patent Application Publication
(10) Pub. No.: US 2023/0157579 A1
(43) Pub. Date: May 25, 2023

(54) DETECTION SYSTEM, WALKING EXERCISE SYSTEM, DETECTION METHOD AND STORAGE MEDIUM

(71) Applicant: TOYOTA JIDOSHA KABUSHIKI KAISHA, Toyota-shi (JP)

(72) Inventor: Yo SATO, Miyoshi-shi (JP)

(21) Appl. No.: 17/938,363

(22) Filed: Oct. 6, 2022

(30) Foreign Application Priority Data
Nov. 19, 2021 (JP) 2021-188343

(51) Int. Cl. A61B 5/103; A61B 5/00; A61H 1/02; A63B 22/02

(52) U.S. Cl. CPC A61B 5/1038; A61B 5/725; A61H 1/0262; A63B 22/02; A61B 2562/0252; A61B 2505/09

(57) ABSTRACT

A detection system includes an acquisition unit, a calculation unit, a determination unit and a correction unit. The acquisition unit acquires measurement information from a load distribution sensor that detects a distribution of a load received from a sole of a subject. The calculation unit calculates a total load value of a sole region corresponding to the position of a sole of one leg of the subject, based on the measurement information. The determination unit determines an action state of the one leg based on the total load value. The correction unit starts to offset the total load value using an offset filter that decreases an offset amount with time elapse, in response to a determination that the action state is a first action state where the total load value tends to increase and is equal to or larger than a preset determination value.

### CROSS-REFERENCE TO RELATED APPLICATION

This application claims priority to Japanese Patent Application No. 2021-188343 filed on Nov. 19, 2021, incorporated herein by reference in its entirety.

### BACKGROUND

### 1. Technical Field

The present disclosure relates to a detection system, a walking exercise system, a detection method and a storage medium.

### 2. Description of Related Art

There has been developed a walking exercise system in which a rehabilitation patient performs exercise for walking action. In the walking exercise system, the load distribution of an exerciser is measured by a load distribution sensor installed in a treadmill. For example, WO2006/106714 discloses a pressure distribution detection device including two kinds of loop electrode groups, elastic bodies on the loop electrode groups, and conductive substances on the elastic bodies. The walking exercise system measures the walking state of the exerciser based on a load value that is obtained from a measurement result, and aids the motion of the joint of the exerciser.

### SUMMARY

For example, in the case where the load value exceeds a control determination value and then falls below the control determination value again, the walking exercise system detects a state where the exerciser has started to weaken stepping force, and aids the extension of the joint of the exerciser. However, in the case where the pressure distribution detection device described in WO2006/106714 is used in the walking exercise system, stress is gradually transmitted in an elastic member, so that there is a gap between the output of the load distribution sensor and the actually applied load. Thereby, there is a problem in that the state where the exerciser has started to weaken the stepping force can be falsely detected.

The present disclosure has been made for solving this problem, and provides a detection system, a walking exercise system, a detection method and a storage medium that improve the detection accuracy of the action state of the leg.

A detection system according to an aspect of the present disclosure includes an acquisition unit, a calculation unit, a determination unit and a correction unit. The acquisition unit acquires measurement information from a load distribution sensor, the load distribution sensor detecting a distribution of a load that is received from a sole of a subject. The calculation unit calculates a total load value of a sole region based on the measurement information, the sole region corresponding to the position of a sole of one leg of the subject. The determination unit determines an action state of the one leg based on the total load value. The correction unit starts to offset the total load value using an offset filter, in response to a determination that the action state is a first action state, the offset filter decreasing an offset amount with time elapse, the first action state being a state where the total load value tends to increase and is equal to or larger than a preset determination value. Thereby, the detection system can improve the detection accuracy of the action state of the leg. Further, the determination unit may determine that the action state is a second action state, in a case where the offset total load value tends to decrease and is smaller than the determination value. Thereby, it is possible to avoid of the false detection of the state where the exerciser has started to weaken the stepping force.

The correction unit may generate the offset filter, based on an output characteristic of the load distribution sensor to an input load and an attribute of the subject. Thereby, it is possible to reflect the output characteristic of the load distribution sensor that corresponds to a pattern of the input load that is estimated from the attribute of the subject, in the offset amount.

Particularly, the correction unit may generate the offset filter, based on the output characteristic of the load distribution sensor to the input load and the body weight of the subject. By estimating the input load from the body weight value, it is possible to generate the offset filter easily and accurately.

Further, the correction unit may generate the offset filter, based on a state of the sole of the subject when the sole begins to be grounded, in a case where it is determined that the action state is the first action state. Thereby, the correction unit can execute an offset correction appropriate for the walking state of the exerciser.

The determination unit may determine that the action sate is the first action state, in a case where the area of the sole region is equal to or larger than a predetermined area threshold.

A walking exercise system according to an aspect of the present disclosure includes: a control device configured to control extension of a leg robot worn on at least one leg of a subject, based on an action state of the leg of the subject; a load distribution sensor configured to detect a distribution of a load that is received from a sole of the subject; and a detection device. The detection device includes an acquisition unit, a calculation unit, a determination unit and a correction unit. The acquisition unit acquires measurement information from the load distribution sensor. The calculation unit calculates a total load value of a sole region based on the measurement information, the sole region corresponding to the position of a sole of one leg of the subject. The determination unit determines an action state of the one leg based on the total load value. The correction unit starts to offset the total load value using an offset filter, in response to a determination that the action state is a first action state, the offset filter decreasing an offset amount with time elapse, the first action state being a state where the total load value tends to increase and is equal to or larger than a preset determination value. Thereby, the walking exercise system can improve the detection accuracy of the action state of the leg. Further, the determination unit may determine that the action state is a second action state, in a case where the offset total load value tends to decrease and is smaller than the determination value. Thereby, it is possible to avoid of the false detection of the state where the exerciser has started to weaken the stepping force.

The control device may control the extension of the leg robot in response to a detection of the second action state.

A detection method according to an aspect of the present disclosure includes: a step of acquiring measurement information from a load distribution sensor, the load distribution sensor detecting a distribution of a load that is received from a sole of a subject; a step of calculating a total load value of a sole region based on the measurement information, the sole region corresponding to the position of a sole of one leg of the subject; a step of determining an action state of the one leg based on the total load value; and a step of starting to offset the total load value using an offset filter, in response to a determination that the action state is a first action state, the offset filter decreasing an offset amount with time elapse, the first action state being a state where the total load value tends to increase and is equal to or larger than a preset determination value. Thereby, it is possible to improve the detection accuracy of the action state of the leg.

A storage medium storing a program according to an aspect of the present disclosure causes a computer to execute a detection method. The detection method includes: a step of acquiring measurement information from a load distribution sensor, the load distribution sensor detecting a distribution of a load that is received from a sole of a subject; a step of calculating a total load value of a sole region based on the measurement information, the sole region corresponding to the position of a sole of one leg of the subject; a step of determining an action state of the one leg based on the total load value; and a step of starting to offset the total load value using an offset filter, in response to a determination that the action state is a first action state, the offset filter decreasing an offset amount with time elapse, the first action state being a state where the total load value tends to increase and is equal to or larger than a preset determination value. Thereby, it is possible to improve the detection accuracy of the action state of the leg.

The present disclosure can provide a detection system, a walking exercise system, a detection method and a storage medium that improve the detection accuracy of the action state of the leg.

### BRIEF DESCRIPTION OF THE DRAWINGS

Features, advantages, and technical and industrial significance of exemplary embodiments of the disclosure will be described below with reference to the accompanying drawings, in which like signs denote like elements, and wherein:

FIG. 1 is a schematic perspective view of a walking exercise system according to an embodiment;

FIG. 2 is a schematic perspective view showing an exemplary configuration of a walking exercise device;

FIG. 3 shows a lateral view and top view of a treadmill according to the embodiment;

FIG. 4 is a diagram showing an example of the output characteristic of a load distribution sensor to an input load;

FIG. 5 is a diagram showing an example of the output characteristic of the load distribution sensor when a load is received from the sole of one leg of an exerciser during walking;

FIG. 6 is a diagram for describing an example of an offset amount according to the embodiment;

FIG. 7 is a block diagram showing a schematic configuration of a detection device according to the embodiment;

FIG. 8 is a flowchart showing a procedure of a detection method according to the embodiment;

FIG. 9 is a diagram showing an example of an offset filter according to the embodiment;

FIG. 10 is a diagram showing another example of the offset filter according to the embodiment;

FIG. 11 is a diagram for describing an estimation process for a sole region according to the embodiment; and

FIG. 12 is a schematic configuration diagram of a computer that is used as the detection device and a system control unit according to the embodiment.

### DETAILED DESCRIPTION OF EMBODIMENTS

The present disclosure will be described below with an embodiment. The disclosure according to the claims is not limited to the embodiment described below. Further, all configurations described in the embodiment are not essential as means for solving the problem. For clear explanations, in the following description and the drawings, omission and simplification are performed when appropriate.

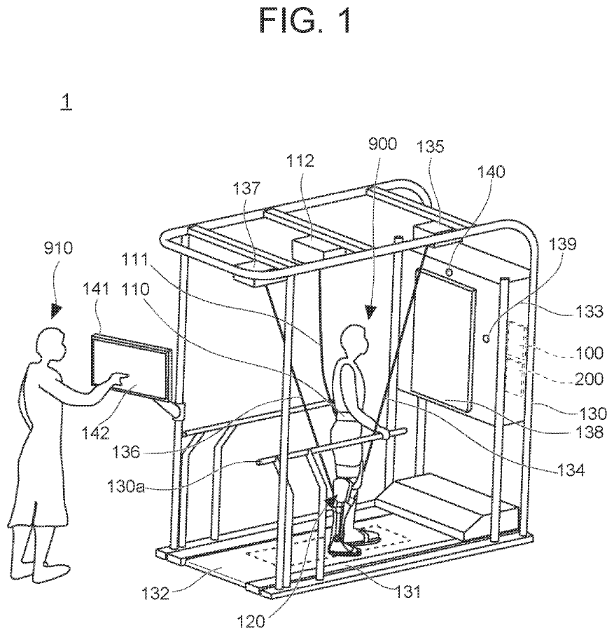

FIG. 1 is a schematic perspective view of a walking exercise system 1 according to the embodiment. The walking exercise system 1 is an example of a system to which a detection device (also referred to as a detection system) according to the embodiment can be applied. The walking exercise system 1 is a system in which walking exercise is performed by an exerciser 900 that is a hemiplegia patient suffering the paralysis of one leg. The exerciser 900 is also referred to as a subject. The up-down direction, right-left direction and front-rear direction in the following description are directions on the basis of the orientation of the exerciser 900.

The walking exercise system 1 mainly includes a control panel 133 that is attached to a frame 130 forming a whole skeleton, a treadmill 131 on which the exerciser 900 walks, and a walking aid device 120 that is worn on at least one leg of the exerciser 900. In the embodiment, the at least one leg is an affected leg that is a leg portion on the paralysis side of the exerciser 900.

The frame 130 is provided so as to stand on the treadmill 131 that is installed on a floor surface. The treadmill 131 rotates a ring-shaped belt 132 by an unillustrated motor. Thereby, the belt 132 runs along an orbit. The treadmill 131 is a device that encourages the walking of the exerciser 900. When performing walking exercise, the exerciser 900 gets on the belt 132, and tries walking action on a walking surface formed on the belt 132.

The frame 130 supports the control panel 133, an exercise monitor 138 and a voice output unit 139. The control panel 133 contains a detection device 100 and a system control unit 200. The detection device 100 is a computer device that detects the action state of the leg of the exerciser 900 walking on the walking surface, from a measurement result of a sensor. The system control unit 200, which is also referred to as a control device, is a computer device that controls the sensor and the motor. For example, the system control unit 200 controls the extension of the walking aid device 120, based on the action state of the leg of the exerciser 900 that is detected by the detection device 100.

The exercise monitor 138 is a display device that presents information relevant to the exercise and the measurement, to the exerciser 900. For example, the exercise monitor 138 is a liquid crystal panel. The exercise monitor 138 is installed such that the exerciser 900 can visually recognize the exercise monitor 138 while walking on the belt 132 of the treadmill 131.

The voice output unit 139 outputs information relevant to the exercise and the measurement, by voice, and informs the exerciser 900. For example, the voice output unit 139 is a speaker. The voice output unit 139 is installed at such a position that the exerciser 900 can hear the voice while walking on the belt 132 of the treadmill 131.

Further, the frame 130 supports a front-side pulling unit 135 near a head upper front portion of the exerciser 900, supports a harness pulling unit 112 near a head upper portion of the exerciser 900, and supports a rear-side pulling unit 137 near a head upper rear portion of the exerciser 900. Further, the frame 130 may include a rail 130a that is grasped by the exerciser 900.

A camera 140 is a front camera unit that picks up the image of the exerciser 900 at a field angle that makes it possible to recognize the gait of the exerciser 900 from the front. The camera 140 may include a side camera unit that picks up the image of the exerciser 900 at a field angle that makes it possible to recognize the gait of the exerciser 900 from a side. The camera 140 in the embodiment includes a set of a lens and an image pickup element. The lens has a field angle that makes it possible to capture the whole body including a head portion of the exerciser 900 that stands on the belt 132. The image pickup element is a CMOS image sensor, for example, and converts an optical image formed on an image forming surface, into an image signal. The camera 140 is installed at the vicinity of the exercise monitor 138, so as to be oriented to the exerciser 900. In the case where the camera 140 includes the side camera unit, the side camera unit may be installed on the rail 130a, so as to capture the exerciser 900 from the side.

One end of a front-side wire 134 is joined to a wind-up mechanism of the front-side pulling unit 135, and the other end is joined to the walking aid device 120. The wind-up mechanism of the front-side pulling unit 135 turns an unillustrated motor on or off in accordance with an instruction from the system control unit 200, and thereby winds up or pays out the front-side wire 134 depending on the motion of the affected leg. Similarly, one end of a rear-side wire 136 is joined to a wind-up mechanism of the rear-side pulling unit 137, and the other end is joined to the walking aid device 120. The wind-up mechanism of the rear-side pulling unit 137 turns an unillustrated motor on or off in accordance with an instruction from the system control unit 200, and thereby winds up or pays out the rear-side wire 136 depending on the motion of the affected leg. By such a cooperative action of the front-side pulling unit 135 and the rear-side pulling unit 137, the load from the walking aid device 120 is cancelled such that the load does not become a burden on the affected leg, and further a motion start action of the affected leg is assisted depending on the level of setting.

An operator 910 that is an exerciser aid person sets the assist level to a high level, for an exerciser that suffers a severe paralysis. The operator 910 is a physical therapist or doctor that has the authority to select, alter and add setting items for the walking exercise system 1. When the assist level is set to a high level, the front-side pulling unit 135 winds up the front-side wire 134 with a relatively great force, at the same timing as the start of the motion of the affected leg. When the exercise advances and the assist becomes unnecessary, the operator sets the assist level to the lowest level. When the assist level is set to the lowest level, the front-side pulling unit 135 winds up the front-side wire 134 with a force allowing the self-weight of the walking aid device 120 to be cancelled, at the same timing as the start of the motion of the affected leg.

The walking exercise system 1 includes a safety device that has a safety outfit 110, a harness wire 111 and the harness pulling unit 112 as main constituent elements. The safety outfit 110 is a belt that is wound around an abdominal portion of the exerciser 900, and is fixed to a lumbar portion by a hook-and-loop fastener, for example. The harness wire 111 is a wire that has one end joined to the safety outfit 110 and that has the other end joined to a wind-up mechanism of the harness pulling unit 112. The wind-up mechanism of the harness pulling unit 112 turns an unillustrated motor on or off, and thereby winds up or pays out the harness wire 111. By this configuration, in the case where the posture of exerciser 900 is greatly lost, the safety device winds up the harness wire 111 in accordance with an instruction from the system control unit 200 that detects the motion, and thereby supports the upper body of the exerciser 900 with the safety outfit 110.

The management monitor 141 is a display device that is attached to the frame 130 and that is monitored and operated by the operator 910. The management monitor 141 is a liquid crystal panel, for example, and a touch panel is superposed on a surface of the management monitor 141, as an example of an input unit 142. The management monitor 141 presents various menu items relevant to setting for the exercise and the measurement, various parameter values at times of the exercise and the measurement, measurement results at the time of the exercise. The input unit 142 may be a keyboard or the like, instead of the touch panel. Further, the operator 910 selects, alters or adds setting items through the input unit 142. Further, the management monitor 141 is installed at such a position that the exerciser 900 cannot visually recognize the display on the management monitor 141 at an exercise trial position on the treadmill 131. A support unit that supports the management monitor 141 may include a rotation mechanism that inverts the display surface. In this case, the operator 910 can purposely cause the exerciser 900 to see the display screen.

The walking aid device 120 is worn on the affected leg of the exerciser 900, and aids the walking of the exerciser 900 by reducing the load for extension and flexion at the knee joint of the affected leg. The walking aid device 120 sends data that is relevant to leg movement and that is obtained by walking exercise, to the system control unit 200, and drives a joint portion in accordance with an instruction from the system control unit 200. The walking aid device 120 may be connected with a hip joint (a connection member including a rotation portion) attached to the safety outfit 110 that is a part of a fall prevention harness device, through a wire or the like.

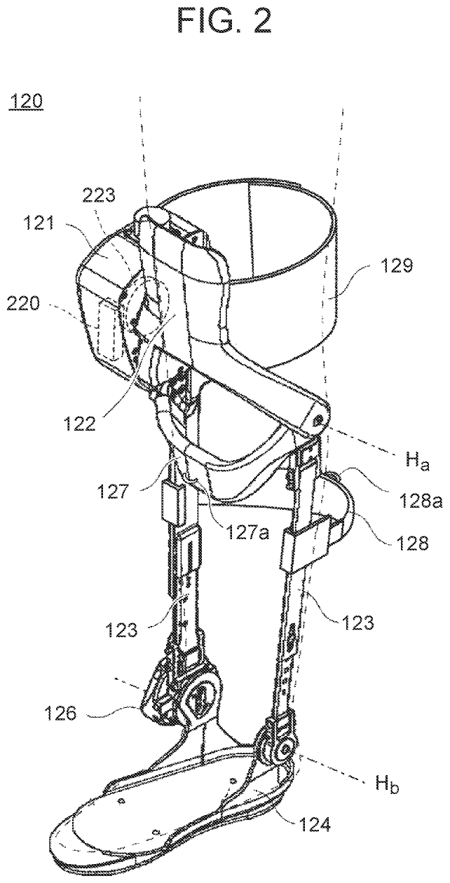

FIG. 2 is a schematic perspective view showing an exemplary configuration of the walking aid device 120. The walking aid device 120 mainly includes a control unit 121 and a plurality of frames that supports portions of the affected leg. The walking aid device 120 is also referred to as a leg robot.

The control unit 121 includes an aid control unit 220 that controls the walking aid device 120, and includes an unillustrated motor that generates a drive force for aiding the extension movement and flexion movement of the knee joint. The frames that supports the portions of the affected leg includes an upper leg frame 122, and lower leg frames 123 pivotally joined to the upper leg frame 122. Further, the frames include a flat foot frame 124 pivotally joined to the lower leg frames 123, a front-side joining frame 127 for joining the front-side wire 134, and a rear-side joining frame 128 for joining the rear-side wire 136.

The upper leg frame 122 and the lower leg frames 123 relatively pivot around an illustrated hinge shaft Ha. The motor of the control unit 121 rotates in accordance with an instruction from the aid control unit 220, and gives force such that the upper leg frame 122 and the lower leg frames 123 are relatively opened around the hinge shaft Ha or gives force such that the upper leg frame 122 and the lower leg frames 123 are relatively closed around the hinge shaft Ha. An angle sensor 223 contained in the control unit 121 is a rotary encoder, for example, and detects the angle between the upper leg frame 122 and the lower leg frames 123 around the hinge shaft Ha. The lower leg frames 123 and the flat foot frame 124 relatively pivot around an illustrated hinge shaft Hb. The angle range of the relative pivoting is adjusted by an adjustment mechanism 126 in advance.

The front-side joining frame 127 is provided so as to extend in the right-left direction on the front side of the upper leg and to connect both ends to the upper leg frame 122. Further, on the front-side joining frame 127, a joining hook 127a for joining the front-side wire 134 is provided near the center in the right-left direction. The rear-side joining frame 128 is provided so as to extend in the right-left direction on the rear side of the lower leg and to connect both ends to the lower leg frames 123 each of which extends in the up-down direction. Further, on the rear-side joining frame 128, a joining hook 128a for joining the rear-side wire 136 is provided near the center in the right-left direction.

The upper leg frame 122 includes an upper leg belt 129. The upper leg belt 129 is a belt provided integrally with the upper leg frame, and fixes the upper leg frame 122 to an upper leg portion of the affected leg while being wound around the upper leg portion. Thereby, the whole of the walking aid device 120 is prevented from deviating from the leg portion of the exerciser 900.

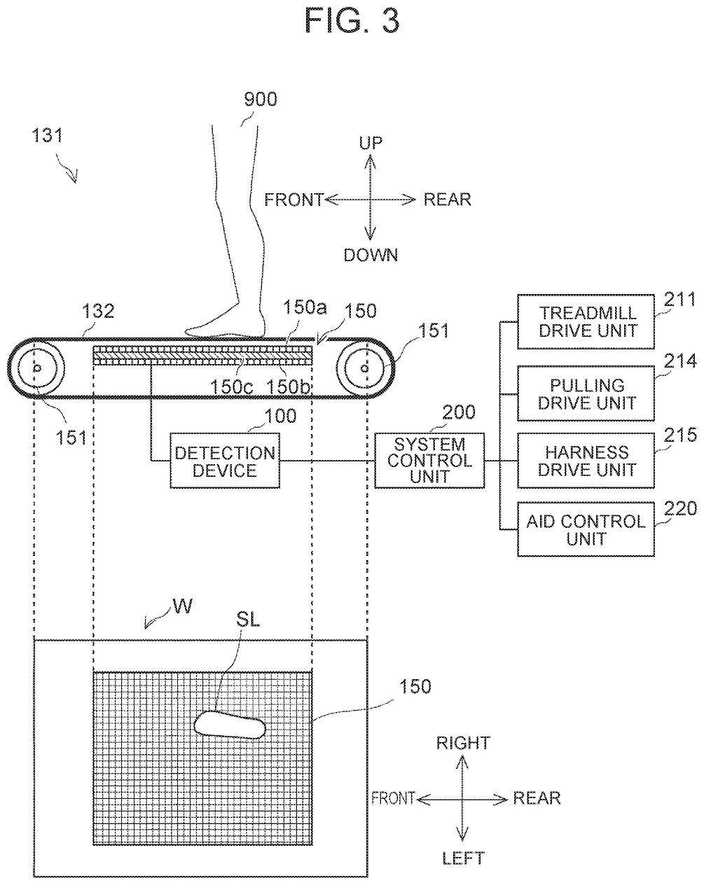

FIG. 3 shows a lateral view and top view of the treadmill according to the embodiment. The treadmill 131 includes at least the ring-shaped belt 132, pulleys 151 and an unillustrated motor.

Further, a load distribution sensor 150 is disposed on the inside of the belt 132, that is, on the opposite side of the surface of the belt 132 on which the exerciser 900 gets. The load distribution sensor 150 is fixed to the treadmill 131, so as not to be moved by the movement of the belt 132.

The load distribution sensor 150 is a load distribution sensor sheet that has a plurality of pressure detection points. The plurality of pressure detection points is disposed in a matrix manner, parallel to a walking surface W (placement surface) that supports the sole of the exerciser 900 in the standing state. Further, the load distribution sensor 150 is disposed at a center portion of the walking surface W in the right-left direction orthogonal to a walking front-rear direction. The walking front-rear direction is a direction that is parallel to the running direction of the belt 132. The load distribution sensor 150 can detect the magnitudes and distribution of vertical loads that are received from the sole of the exerciser 900, by using output values of the plurality of pressure detection points. Thereby, through the belt 132, the load distribution sensor 150 detects the position of a grounding region (sole region) SL of the sole of the exerciser 900 in the standing state, and the distribution of loads that are received from the sole of the exerciser 900. The position of the sole region SL is also referred to as a standing position or stepping position of the exerciser 900.

The load distribution sensor 150 is connected to the detection device 100. The detection device 100 acquires load distribution information from the load distribution sensor 150, as measurement information, and measures the action state of the leg of the exerciser 900 based on the load distribution information. For example, the action state of the leg is a state where the stepping is started, a state where the stepping is maximized, or a state where the force starts to be weakened. The detection device 100 is connected to the system control unit 200 by wire or by wireless, and outputs the measured action state to the system control unit 200.

The system control unit 200 controls various drive units, based on the action state of the leg that is acquired from the detection device 100. For example, the system control unit 200 is connected to a treadmill drive unit 211, a pulling drive unit 214, a harness drive unit 215, and the aid control unit 220 of the walking aid device 120, by wire or by wireless. The system control unit 200 sends drive signals to the treadmill drive unit 211, the pulling drive unit 214 and the harness drive unit 215, and sends a control signal to the aid control unit 220.

The treadmill drive unit 211 includes the above-described motor that rotates the belt 132 of the treadmill 131, and a drive circuit for the motor. The system control unit 200 executes the rotation control of the belt 132, by sending the drive signal to the treadmill drive unit 211. For example, the system control unit 200 adjusts the rotation speed of the belt 132, depending on the walking speed set by the operator 910. Alternatively, the system control unit 200 adjusts the rotation speed of the belt 132, depending on the action state of the leg of the exerciser 900 that is output from the detection device 100.

The pulling drive unit 214 includes a motor that is provided in the front-side pulling unit 135 and that pulls the front-side wire 134, and a drive circuit for the motor, and includes a motor that is provided in the rear-side pulling unit 137 and that pulls the rear-side wire 136, and a drive circuit for the motor. The system control unit 200 controls each of the wind-up of the front-side wire 134 and the wind-up of the rear-side wire 136, by sending the drive signal to the pulling drive unit 214. Further, in addition to the wind-up action, the system control unit 200 controls the pulling force of each wire, by controlling the drive torque of the motor. Furthermore, for example, the system control unit 200 assists the action of the affected leg, by identifying the timing of the switching of the affected leg from a leg standing state to a leg idling state based on the action state of the leg of the exerciser 900 that is output from the detection device 100 and increasing or decreasing the pulling force of each wire in synchronization with the timing.

The harness drive unit 215 includes a motor that is provided in the harness pulling unit 112 and that pulls the harness wire 111, and a drive circuit for the motor. The system control unit 200 controls the wind-up of the harness wire 111 and the pulling force of the harness wire 111, by sending the drive signal to the harness drive unit 215. For example, in the case where the fall of the exerciser 900 is predicted, the system control unit 200 prevents the fall of the exerciser 900 by winding up the harness wire 111 by a certain amount.

The aid control unit 220 is a microprocessor unit (MPU), for example, and executes the control of the walking aid device 120 by executing a control program that is given from the system control unit 200. Further, the aid control unit 220 gives notice of the state of the walking aid device 120, to the system control unit 200. Further, the aid control unit 220 executes the control of the walking aid device 120, as exemplified by the activation and stop of the walking aid device 120, in response to a command from the system control unit 200.

The aid control unit 220 sends a drive signal to a joint drive unit that includes the motor of the control unit 121 and a drive circuit for the motor, and thereby gives force such that the upper leg frame 122 and the lower leg frames 123 are relatively opened around the hinge shaft Ha or gives force such that the upper leg frame 122 and the lower leg frames 123 are relatively closed around the hinge shaft Ha. By this action, the extension action and flexion action of the knee are assisted, and a buckling is prevented. The aid control unit 220 receives a detection signal from an angle sensor (not illustrated) that detects the angle between the upper leg frame 122 and the lower leg frames 123 around the hinge shaft Ha, and computes the opening angle of the knee joint.

In the load distribution sensor 150, an elastic sheet 150c is inserted between two electrode sheets 150a, 150b that face each other. The elastic sheet 150c is an elastic member composed of silicon sponge sheet, urethane foam or the like. The elastic sheet 150c has a property of slowly transmitting stress and gradually deforming when an external force is given, because of influence of viscosity.

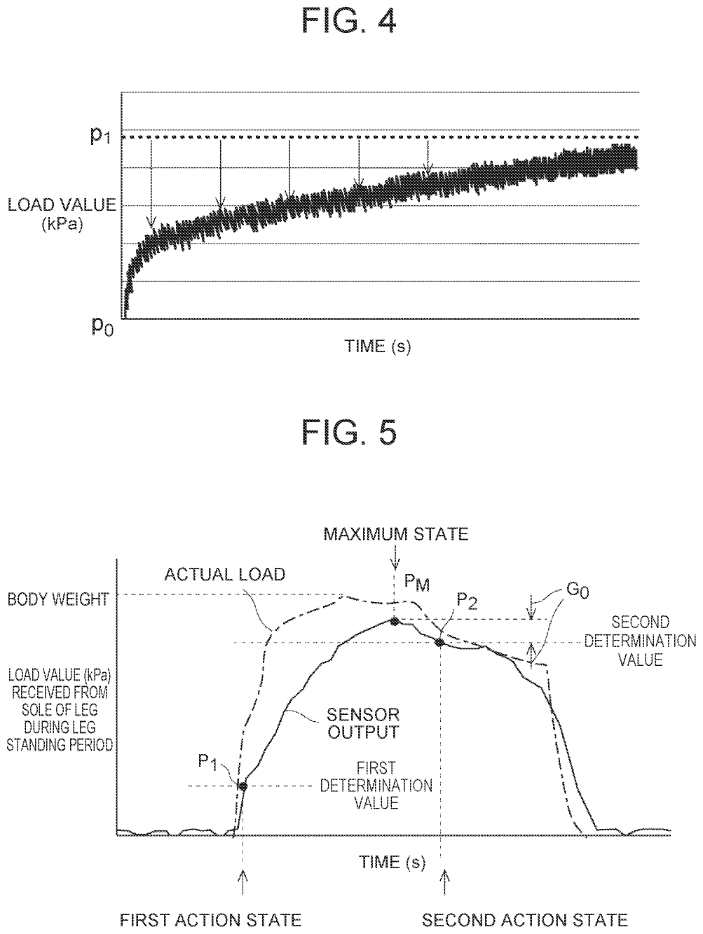

FIG. 4 is a diagram showing an example of the output characteristic of the load distribution sensor 150 to the input load. The figure shows a temporal change in the output value of the load distribution sensor 150 when an external force (input load) having a certain load value p₁ (kPa) is continuously applied to the load distribution sensor 150. As shown in the figure, at a time point when the external force begins to be applied, the output value of the load distribution sensor 150 is lower than the load value of the external force that is actually being applied. This is because the stress is transmitted in the load distribution sensor 150 with a delay due to the existence of the elastic sheet 150c included in the load distribution sensor 150, so that the output of the load distribution sensor 150 is delayed. However, the output value of the load distribution sensor 150 asymptotically becomes closer to the load value pi of the external force that is being applied, with time elapse. That is, the difference between the output value of the load distribution sensor 150 and the load value p₁ of the external force becomes smaller with time elapse. Then, after a predetermined time elapses from the start of the application of the external force, the output value of the load distribution sensor 150 is stabilized at a value comparable to the load value p₁ that is being applied.

FIG. 5 is a diagram showing an example of the output characteristic of the load distribution sensor 150 when a load is received from the sole of one leg of the exerciser 900 during walking. In the embodiment, the one leg is an affected leg. In the figure, the ordinate indicates the load value (kPa) that is received from the sole of the leg during a leg standing period, and the abscissa indicates time (s).

A broken line indicates a load value that is actually applied to the load distribution sensor 150 from the sole of the leg of the exerciser 900 during the leg standing period from landing to departing (“actual load”). In the “actual load”, the load value gradually increase from the start of the stepping, and the load value reaches the maximum at a certain time point. At this time point, the total body weight is supported by the leg during the leg standing period, and therefore the maximum value of the load value is nearly equal to the body weight of the exerciser 900. Thereafter, in a leg standing latter period, the exerciser 900 starts to gradually weaken the force, and therefore the load value gradually decreases. Then, the exerciser 900 causes the leg to depart from the ground, resulting in the transition to a leg idling period.

A solid line indicates a load value that is received from the sole of the leg during the leg standing period of the exerciser 900 and that is calculated based on the output of the load distribution sensor 150 (“sensor output”). The “sensor output” is obtained by extracting the load value that is received from the sole of the leg during the leg standing period, from the output of the load distribution sensor 150. Specifically, the “sensor output” is obtained by calculating the sum of the load values output from the pressure detection points within the sole region SL of the leg during the leg standing period.

The “sensor output” increases in a leg standing beginning period, and when the “sensor output” exceeds a first determination value (point P₁), the detection device 100 detects a first action state. The first action state is a state where the stepping is started. Then, when the “sensor output” reaches the maximum (point P_{M}), the detection device 100 detects a maximum state. The maximum state is a state where the stepping is maximized. Further, a second determination value is a value that is larger than the first determination value and smaller than the body weight value of the exerciser 900. After the point PM, the “sensor output” gradually decreases, and when the “sensor output” falls below the second determination value (point P₂), the detection device 100 detects a second action state. The second action state is a state where the force starts to be weakened. In response to the detection of the second action state, the system control unit 200 sends the control signal to the aid control unit 220, and controls the extension of the walking aid device 120. Alternatively, in response to the detection of the second action state, the system control unit 200 sends the drive signal to the pulling drive unit 214, and assists the action of the affected leg.

The value of the “sensor output” is lower than the load value that is actually applied, until the output becomes stable, because of the influence of the above-described output characteristic of the load distribution sensor 150. Accordingly, there is a gap between the “sensor output” and the “actual load”. This causes the false detection of the second action state. For solving this problem, in the embodiment, the detection device 100 offsets the “sensor output” by a predetermined amount, from the detection of the first action state. Hereinafter, “offsetting” is sometimes referred to as “correcting” or “performing offset correction”.

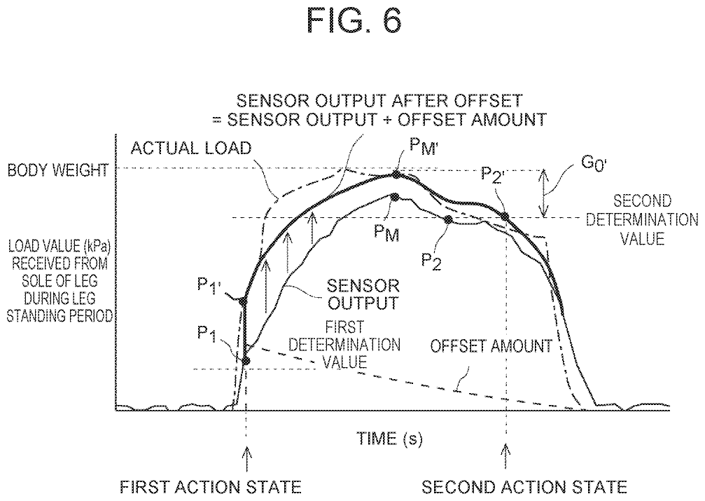

FIG. 6 is a diagram for describing an example of an offset amount according to the embodiment. For example, the detection device 100 offsets the “sensor output”, by adding an offset amount (broken lint) that monotonically decreases with time elapse, to the “sensor output” (thin solid line). In the figure, the “sensor output after offset” is indicated by a thick solid line. The method for deciding the offset amount will be described later. Specifically, in response to the detection of the first action state, the detection device 100 starts the offset of the “sensor output” (point P₁′). Thereby, the “sensor output after offset” becomes closer to the “actual load”, compared to the “sensor output (before offset)”. Accordingly, the error of the detection timing of the second action state (point P₂′) of the “sensor output after offset” is smaller than that of the detection timing of the second action state (point P₂) of the “sensor output (before offset)”. Accordingly, it is possible to avoid the false detection of the second action state, and to improve the detection accuracy.

In the embodiment, the offset amount becomes zero after the elapsed of a predetermined time and therefore the detection device 100 does not need to terminate (end) the offset after the start of the offset. However, the detection device 100 may terminate the offset when the offset amount becomes equal to or smaller than a predetermined value. The predetermined value may be zero, or may be a larger value than zero.

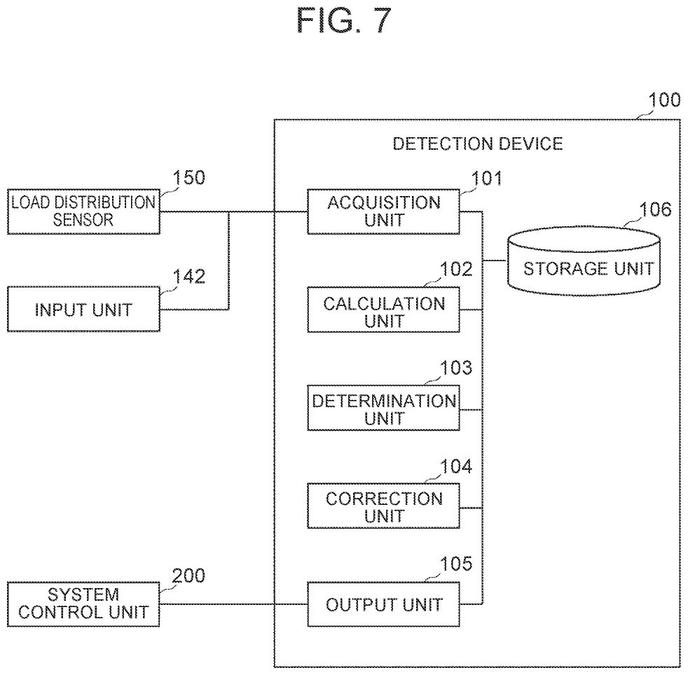

FIG. 7 is a block diagram showing a schematic configuration of the detection device 100 according to the embodiment. The detection device 100 includes an acquisition unit 101, a calculation unit 102, a determination unit 103, a correction unit 104, an output unit 105, and a storage unit 106. The constituent elements of the detection device 100 are connected with each other.

The acquisition unit 101 acquires the load distribution information from the load distribution sensor 150, as the measurement information. For example, the acquisition unit 101 is connected to the load distribution sensor 150, and acquires the load distribution information from the load distribution sensor 150. Then, the acquisition unit 101 supplies the load distribution information of the load distribution sensor 150 to the calculation unit 102.

Further, the acquisition unit 101 is connected to the input unit 142, and acquires basic information for generating an offset filter. The basic information is input from the input unit 142. The offset filter is a filter for deciding an offset amount that is applied at a predetermined time point. In the embodiment, the offset filter has a characteristic in which the offset amount decrease with time elapse. The basic information for generating the offset filter includes at least information indicating the output characteristic of the load distribution sensor 150 to the input load. For example, the information indicating the output characteristic may include a time function for the output value to the input load. Further, the information indicating the output characteristic may include the elastic modulus of the elastic sheet 150c included in the load distribution sensor 150, the viscosity coefficient of the elastic sheet 150c, and the thickness of the elastic sheet 150c. Further, the basic information for generating the offset filter includes attribute information about the exerciser 900, for example, information about the body weight value of the exerciser 900. In addition to or instead of this, the basic information may include other attribute information such as the sex, age, foot length information and rehabilitation stage level of the exerciser 900. Further, the acquisition unit 101 supplies the basic information received from the input unit 142, to the correction unit 104.

Based on the load distribution information, the calculation unit 102 calculates a total load value of the sole region SL corresponding to the position of the sole of one leg of the exerciser 900. Specifically, first, the calculation unit 102 extracts the load values of the pressure detection points within the sole region SL of one leg of the exerciser 900, from the load distribution information. Then, based on the extracted load values, the calculation unit 102 calculates a total load value that is the sum of the loads in the sole region SL. One leg is a leg that is an object of the measurement of the action state, and is also referred to as an object leg. The object leg may be an affected leg. The calculation unit 102 supplies information about the calculated total load value, to the determination unit 103.

The determination unit 103 detects various action states of the object leg of the exerciser 900, based on at least one of the total load value calculated by the calculation unit 102 and a later-described total load value after the offset by the correction unit 104. For example, in the case where the total load value calculated by the calculation unit 102 tends to increase and is equal to or larger than the first determination value, the determination unit 103 determines that the action state of the object leg is the first action state. It may be determined that the total load value tends to increase, when the change amount of total load values based on outputs of the load distribution sensor 150 at consecutive measurement timings has a positive value, or when total load values based on outputs of the load distribution sensor 150 in a predetermined time have a positive correlation. Instead of this, in the case where the area of the sole region SL of the object leg is equal to or larger than a predetermined area threshold, the determination unit 103 may determine that the action state of the object leg is the first action state. That is, in the case where the area of the sole region tends to increase and is equal to or larger than the predetermined area threshold, the determination unit 103 may determine that the action state of the object leg is the first action state. It may be determined that the area of the sole region SL tends to increase, when the change amount of areas of the sole region SL of the object leg at consecutive measurement timings has a positive value, or when areas of the sole region SL of the object leg in a predetermined time have a positive correlation. Needless to say, in the first action state, the total load value calculated by the calculation unit 102 is smaller than the second determination value.

In the case where the total load value calculated by the calculation unit 102 reaches the maximum, the determination unit 103 determines that the action state of the object leg is the maximum state. For example, in the case where the tendency of the total load value calculated in the calculation unit 102 changes from an increasing tendency to a decreasing tendency, the determination unit 103 may determine that the total load value calculated by the calculation unit 102 has reached the maximum.

Further, for example, in the case where the total load value after the offset correction by the correction unit 104 tends to decrease and is smaller than the second determination value, the determination unit 103 determines that the action state of the object leg is the second action state. The total load value after the offset correction by the correction unit 104 is equal to the offset total load value in the case where the offset correction by the correction unit 104 is performed, and is equal to the total load value calculated by the calculation unit 102 in the case where the offset correction by the correction unit 104 is terminated. It may be determined that the total load value tends to decrease, when the change amount of total load values based on outputs of the load distribution sensor 150 at consecutive measurement timings has a negative value, or when total load values based on outputs of the load distribution sensor 150 in a predetermined time have a negative correlation. The determination unit 103 supplies a determination result (detection result) to the output unit 105.

The correction unit 104 generates the offset filter based on the basic information for generating the offset filter. For example, the correction unit 104 generates the offset filter based on the information indicating the output characteristic of the load distribution sensor 150 to the input load and the attribute information about the exerciser 900. The output characteristic of the load distribution sensor 150 changes depending on the pattern of the input load. Accordingly, by this configuration, it is possible to reflect the output characteristic of the load distribution sensor 150 depending on the pattern of the input load that is estimated from the attribute of the exerciser 900, in the offset amount. In the embodiment, the correction unit 104 generates the offset filter based on the information indicating the output characteristic of the load distribution sensor 150 to the input load and the body weight value of the exerciser 900. By estimating the input load from the body weight value, the correction unit 104 can generate the offset filter easily and accurately.

Then, the correction unit 104 offsets the total load value, using the generated offset filter. Specifically, in response to the determination that the action state of the object leg is the first action state, the correction unit 104 starts the offset for the total load value calculated by the calculation unit 102, using the offset filter. Thereby, it is possible to cause the total load value to be close to the actual load, and therefore, for example, it is possible to avoid the false detection of the second action state where the force starts to be weakened, so that it is possible to improve the detection accuracy of the action state.

The output unit 105 outputs the control signal based on the detection result supplied from the determination unit 103, to the system control unit 200. In the embodiment, in the case where the second action state is detected, the output unit 105 outputs a control signal indicating the detection of the second action state, to the system control unit 200. However, without being limited to this, also in the case where the first action state or the maximum state is detected, the output unit 105 may output a control signal corresponding to the action state, to the system control unit 200.

The storage unit 106 is a storage medium in which information necessary for processing in the detection device 100 and generated information are stored.

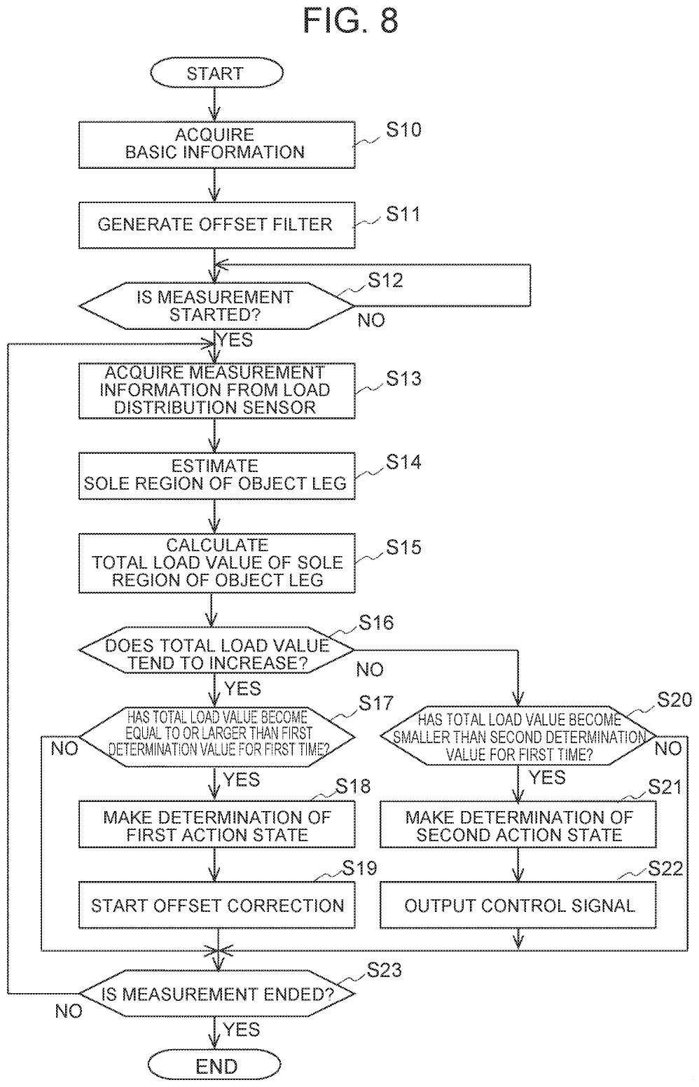

FIG. 8 is a flowchart showing a procedure of a detection method according to the embodiment. First, the acquisition unit 101 acquires the basic information from the exerciser 900 or the operator 910 through the input unit 142 (step S10). Then, the correction unit 104 generates the offset filter based on the basic information (step S11).

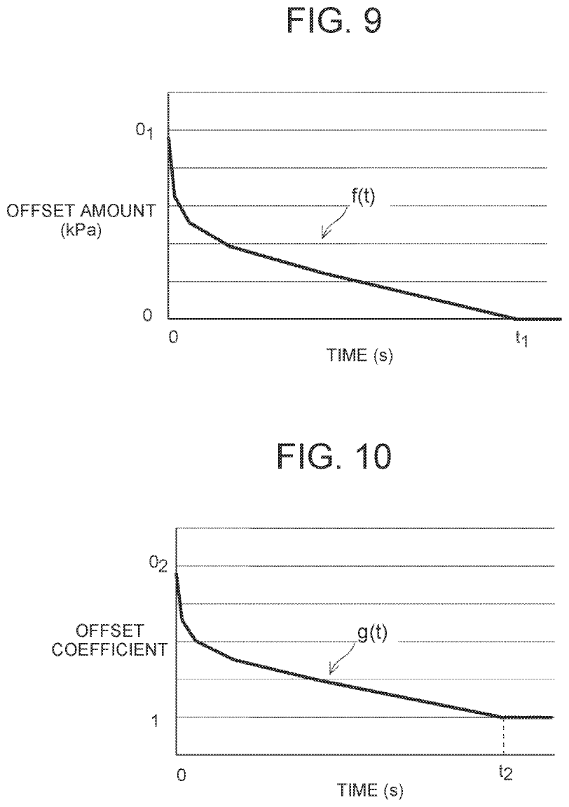

FIG. 9 is a diagram showing an example of the offset filter according to the embodiment. An offset filter f(t) shown in FIG. 9 is a function for time t about the offset amount. The offset filter f(t) may be a filter in which the initial value of the offset amount is o₁ (>0 kPa), in which the offset amount decreases with time elapse, and in which the offset amount becomes zero (kPa) at a preset time t₁. The correction unit 104 may generate the offset filter f(t) by deciding the initial value o₁, the time t₁ and the slope based on the basic information. In the case where the offset filter is a function for time t about the offset amount, the correction unit 104 may execute the offset correction, by adding the value of the offset filter at the current time point, to the total load value.

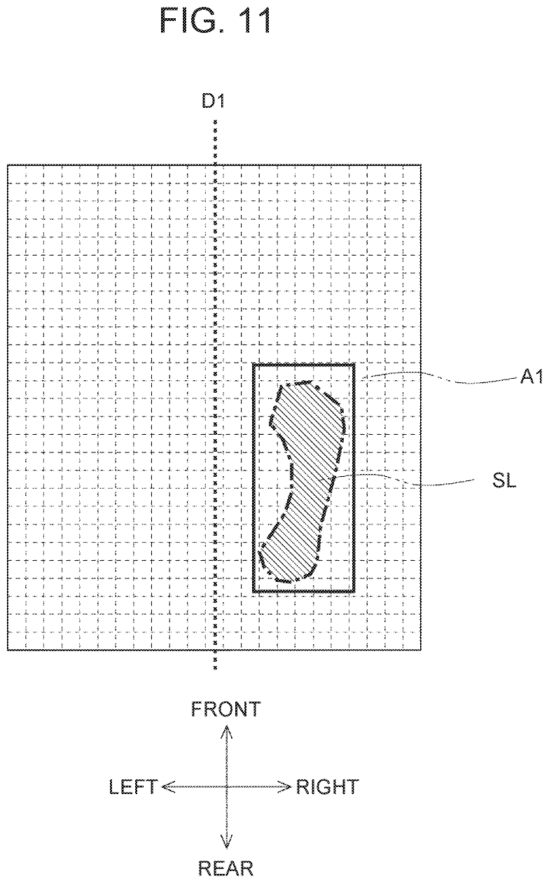

FIG. 10 is a diagram showing another example of the offset filter according to the embodiment. An offset filter g(t) shown in FIG. 10 is a function for time t about an offset coefficient. The offset filter g(t) may be a function in which the initial value of the offset coefficient is o₂ (>1), in which the offset coefficient decreases with time elapse, and in which the offset coefficient becomes 1 (kPa) at a preset time t₂. The correction unit 104 may generate the offset filter g(t) by deciding the initial value o₂, the time t₂ and the slope based on the basic information. In the case where the offset filter is a function for time t about the offset coefficient, the correction unit 104 may execute the offset correction, by multiplying the total load value by the value of the offset filter.

Back to FIG. 8, the description will be continued. The detection device 100 determines whether the measurement is started (step S12). The measurement is started, for example, in the case where the exercise with the walking exercise system 1 is started, or in the case where the detection process with the detection device 100 is started by an operation from the operator 910. The detection device 100 repeats the process shown in step S12, until it is determined that the measurement is started, and in the case where it is determined that the measurement is started (YES in step S12), the detection device 100 causes the process to proceed to step S13.

The acquisition unit 101 acquires the load distribution information from the load distribution sensor 150, as the measurement information (step S13). The load distribution information includes information indicating load values respectively corresponding to pressure detection points at different positions from each other. Then, the acquisition unit 101 supplies the load distribution information to the calculation unit 102. Next, the calculation unit 102 estimates the sole region SL of the object leg based on the load distribution information (step S14), and extracts the load values in the sole region SL of the object leg. Then, the calculation unit 102 calculates the total load value that is the sum of the extracted load values in the sole region SL of the object leg (step S15).

FIG. 11 is a diagram for describing an estimation process for the sole region SL according to the embodiment. For example, the calculation unit 102 generates a load distribution map shown in FIG. 11, based on position information about the pressure detection points and the load values detected from the pressure detection points. The calculation unit 102 may generate the load distribution map, by extracting position information about load values equal to or larger than a detection threshold, from the load values detected from the pressure detection points. Then, the calculation unit 102 detects the sole region SL based on the load distribution map. Here, suppose that the object leg is the right leg. The calculation unit 102 determines whether the sole region SL is a region of the object leg, based on the position of the sole region SL with respect to a central axis D1 of the load distribution sensor 150 in the right-left direction. For example, the calculation unit 102 calculates the position of the barycenter of the sole region SL, and in the case where the position of the barycenter is on the right side of the center axis D1, the calculation unit 102 determines that the sole region SL is the sole region SL of the object leg. Then, the calculation unit 102 calculates the sum of the load values included in the sole region SL, as the total load value. The region in which load values are extracted is not limited to the sole region SL, and may be a predetermined region A1 that contains the sole region SL.

Further, the calculation unit 102 may determine whether the detected sole region SL is the sole region SL of the object leg, based on a photographed image generated by photographing the gait of the exerciser 900 with the camera 140 that is the front camera unit and the side camera unit. For example, in the case where the photographed image shows that the exerciser 900 moves the right leg forward, or in the case where the photographed image shows that the exerciser 900 causes the right leg to be grounded, the calculation unit 102 determines that the detected sole region SL is the sole region SL of the object leg.

The case where the calculation unit 102 detects one sole region SL, that is, the case where the sole of one leg is grounded was described above. However, in the case where the calculation unit 102 detects two sole regions SL, that is, in the case where the soles of both legs are grounded, the calculation unit 102 may estimate the sole region SL of the object leg, based on the relative positions of the two sole regions SL. For example, the calculation unit 102 may set the sole region SL that is of the two sole regions SL and that is on the right side, as the sole region SL of the object leg. Also in this case, the calculation unit 102 may estimate the sole region SL of the object leg based on the photographed image.

Further, since the exerciser 900 walks while the sole of the right leg and the sole of the left leg are alternately grounded, the calculation unit 102 may estimate the sole region SL of the object leg, depending on a walking cycle.

Back to FIG. 8, the description will be continued. In step S16, the determination unit 103 determines whether the total load value tends to increase. In the case where the correction unit 104 is not performing the offset correction, that is, in the case where the offset amount is zero, the total load value is the total load value calculated by the calculation unit 102. Further, in the case where the correction unit 104 is performing the offset correction, that is, in the case where the offset amount is not zero, the total load value is the total load value after the offset. In the case where the determination unit 103 determines that the total load value tends to increase (YES in step S16), the determination unit 103 determines whether the total load value has become equal to or larger than the first determination value for the first time in a first object period (step S17).

The first object period is a period that is in the current walking cycle and during which the total load value tends to increase. In the case where the determination unit 103 determines that the total load value has not yet become equal to or larger than the first determination value in the first object period or that the total load value has become equal to or larger than the first determination value in the first object period in the past (NO is step S17), the determination unit 103 causes the process to proceed to step S23. On the other hand, in the case where the determination unit 103 determines that the total load value has become equal to or larger than the first determination value for the first time in the first object period (YES in step S17), the determination unit 103 determines that the action state of the object leg is the first action state (step S18). Then, the correction unit 104 starts the offset correction for the total load value, using the offset filter (step S19), and causes the process to proceed to step S23.

In the case where the determination unit 103 determines that the total load value does not tend to increase (NO in step S16), the determination unit 103 determines whether the total load value has become smaller than the second determination value for the first time in a second object period (step S20). The second object period is a period that is in the current walking cycle and during which the total load value tends to decrease. In the case where the determination unit 103 determines that the total load value has not yet become smaller than the second determination value in the second object period or that the total load value has become smaller than the second determination value in the second object period in the past (NO in step S20), the determination unit 103 causes the process to proceed to step S23. On the other hand, in the case where the determination unit 103 determines that the total load value has become smaller than the second determination value for the first time in the second object period (YES in step S20), the determination unit 103 determines that the action state of the object leg is the second action state (step S21). Then, the output unit 105 outputs the control signal indicating the detection of the second action state, to the system control unit 200 (step S22), and causes the process to proceed to step S23.

In step S23, the detection device 100 determines whether the measurement is ended. The measurement is ended, for example, in the case where the exercise with the walking exercise system 1 is ended, or in the case where the detection process with the detection device 100 is ended by an operation from the operator 910. The detection device 100 repeats the processes shown in steps S13 to S23, until it is determined that the measurement is ended.

FIG. 8 shows a flow in the case where the detection device 100 does not terminate the offset correction. However, in the case where the detection device 100 terminates the offset correction, the detection device 100 may execute the following process, just before step S23 (in step S19, in step S22, or after step S17 in the case of NO in step S17), for example. In the case where the offset amount is equal to or smaller than a predetermined value, the correction unit 104 of the detection device 100 may terminate the offset correction, and may cause the process to proceed to step S23. On the other hand, in the case where the offset amount is larger than a predetermined value, the correction unit 104 may continue to execute the offset correction, and may cause the process to proceed to step S23.

In this way, with the embodiment, the detection device 100 can improve the detection accuracy of the action state of the leg that is measured, and particularly, can improve the detection accuracy of the timing when the force starts to be weakened.

In the above description, the correction unit 104 generates the offset filter based on the basic information, in step S11 of FIG. 8 before the start of the measurement. However, instead of or in addition to this, the correction unit 104 may generate the offset filter during the measurement. For example, the correction unit 104 may generate the offset filter based on the state of the sole of the exerciser 900 when the sole begins to be grounded, in a period between step S15 and step S16 before it is determined that the action state of the object leg is the first action state. Then, the correction unit 104 may execute the offset correction using the generated offset filter. The state of the sole when the sole begins to be grounded may be a “heel grounding state” in the case where the heel is grounded earlier or a “toe grounding state” in the case where the toe is grounded earlier. The state of the sole when the sole begins to be grounded may be determined by the calculation unit 102, and the information about the state of the sole when the sole begins to be grounded may be supplied from the calculation unit 102 to the correction unit 104. For example, in the case where the calculation unit 102 determines that the sole region SL has expanded in the rearward direction of the walking with time after the detection based on the temporal change in the area of the sole region SL and the running speed of the treadmill 131, the calculation unit 102 may determine that the state of the sole when the sole begins to be grounded is the “toe grounding state”. On the other hand, in the case where the sole region SL has expanded in the forward direction of the walking with time after the detection, the calculation unit 102 may determine that the state of the sole when the sole begins to be grounded is the “heel grounding state”. Offset filters corresponding to the “toe grounding state” and the “heel grounding state” may be different from each other in at least one of the initial value, the slope and the time. Thereby, the correction unit 104 can execute the offset correction appropriate for the walking state of the exerciser 900.

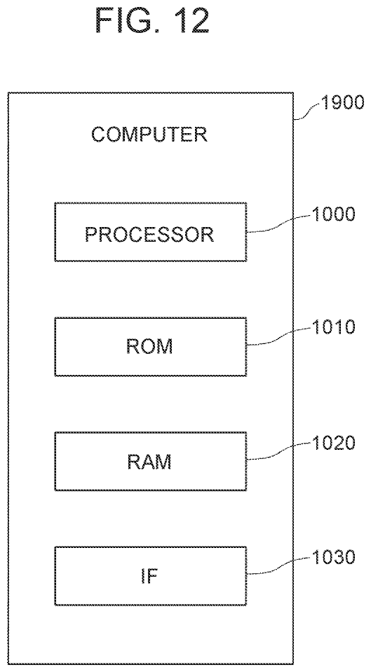

FIG. 12 is a schematic configuration diagram of a computer that is used as the detection device and the system control unit 200 according to the embodiment. A computer 1900 includes a processor 1000, a read only memory (ROM) 1010, a random access memory (RAM) 1020, and an interface (IF) unit 1030, as primary hardware constituents. The processor 1000, the ROM 1010, the RAM 1020 and the interface unit 1030 are connected with each other through a data bus and the like.

The processor 1000 has a function as an arithmetic device that performs control processing, arithmetic processing and the like. The processor 1000 may be a central processing unit (CPU), a graphics processing unit (GPU), a field-programmable gate array (FPGA), a digital signal processor (DSP), an application specific integrated circuit (ASIC) or a combination of them. The ROM 1010 has a function to store control programs, arithmetic programs and the like that are executed by the processor 1000. The RAM 1020 has a function to temporarily store processing data and the like. The interface unit 1030 exchanges signals with the exterior by wire or by wireless. Further, the interface unit 1030 accepts user's operation to input data, and displays information to the user. For example, the interface unit 1030 communicates with the load distribution sensor 150, the input unit 142 and the system control unit 200.

In the above-described example, the programs include commands (or software codes) that causes a computer to execute one or more functions described in the embodiment when being read by the computer. The programs may be stored in various non-transitory computer-readable media, each of which is an example of the ROM 1010, or tangible storage media. Although not limited, examples of the computer-readable media or the tangible storage media include memory technologies such as a random-access memory (RAM), a read-only memory (ROM), a flash memory, a solid-state drive (SSD), optical disk storages such as a CD-ROM, a digital versatile disc (DVD) and a Blu-ray (registered trademark) disk, and magnetic storage devices such as a magnetic cassette, a magnetic tape and a magnetic disk storage. The programs may be sent through transitory computer-readable media or communication media. Although not limited, examples of the transitory computer-readable media or the communication media include an electric propagation signal, an optical propagation signal, an acoustic propagation signal, and another type of propagation signal.

In the above-described embodiment, the computer 1900 is configured by a computer system including a personal computer, a word processor and the like. However, without being limited to this, the computer 1900 can be configured by a server in a local area network (LAN), a host for computer (PC) communication, a computer system connected to the internet, and the like. Further, function distribution may be performed among devices on a network, and the computer 1900 may be configured by the whole of the network. Accordingly, constituent elements of the detection device may be distributed among different devices from each other.

The present disclosure is not limited to the above embodiment, and modifications can be made without departing from the spirit, when appropriate. For example, in the above-described embodiment, the detection device 100 detects the action state of the affected leg as the object leg. However, the detection device 100 may detect the action state of a normal leg. Further, the detection device 100 may detect the action state of each of the right and left legs. In this case, for each leg, the processes shown in steps S13 to S23 of FIG. 8 are executed.

Further, in the above embodiment, the second determination value is larger than the first determination value. However, the second determination value may be equal to the first determination value.

Further, the exerciser 900 may wear the walking aid device 120 on both legs, and may perform exercise. Alternatively, the exerciser 900 does not need to wear the walking aid device 120 on any leg.

What is claimed is:

1. A detection system comprising:
an acquisition unit configured to acquire measurement information from a load distribution sensor, the load distribution sensor detecting a distribution of a load that is received from a sole of a subject;
a calculation unit configured to calculate a total load value of a sole region based on the measurement information, the sole region corresponding to a position of a sole of one leg of the subject;
a determination unit configured to determine an action state of the one leg based on the total load value; and
a correction unit configured to start to offset the total load value using an offset filter, in response to a determination that the action state is a first action state, the offset filter decreasing an offset amount with time elapse, the first action state being a state where the total load value tends to increase and is equal to or larger than a preset determination value.

2. The detection system according to claim 1, wherein the determination unit determines that the action state is a second action state, in a case where the offset total load value tends to decrease and is smaller than the determination value.

3. The detection system according to claim 1, wherein the correction unit generates the offset filter, based on an output characteristic of the load distribution sensor to an input load and an attribute of the subject.

4. The detection system according to claim 3, wherein the correction unit generates the offset filter, based on the output characteristic of the load distribution sensor to the input load and a body weight of the subject.

5. The detection system according to claim 1, wherein the correction unit generates the offset filter, based on a state of the sole of the subject when the sole begins to be grounded, in a case where it is determined that the action state is the first action state.

6. The detection system according to claim 1, wherein the determination unit determines that the action sate is the first action state, in a case where an area of the sole region is equal to or larger than a predetermined area threshold.

7. A walking exercise system comprising:
a control device configured to control extension of a leg robot worn on at least one leg of a subject, based on an action state of the leg of the subject;
a load distribution sensor configured to detect a distribution of a load that is received from a sole of the subject; and
a detection device, wherein
the detection device includes:
an acquisition unit configured to acquire measurement information from the load distribution sensor;
a calculation unit configured to calculate a total load value of a sole region based on the measurement information, the sole region corresponding to a position of a sole of one leg of the subject;
a determination unit configured to determine an action state of the one leg based on the total load value; and
a correction unit configured to start to offset the total load value using an offset filter, in response to a determination that the action state is a first action state, the offset filter decreasing an offset amount with time elapse, the first action state being a state where the total load value tends to increase and is equal to or larger than a preset determination value.

8. The walking exercise system according to claim 7, wherein the determination unit determines that the action state is a second action state, in a case where the offset total load value tends to decrease and is smaller than the determination value.

9. The walking exercise system according to claim 8, wherein the control device controls the extension of the leg robot in response to a detection of the second action state.

10. A detection method comprising:
a step of acquiring measurement information from a load distribution sensor, the load distribution sensor detecting a distribution of a load that is received from a sole of a subject;
a step of calculating a total load value of a sole region based on the measurement information, the sole region corresponding to a position of a sole of one leg of the subject;
a step of determining an action state of the one leg based on the total load value; and
a step of starting to offset the total load value using an offset filter, in response to a determination that the action state is a first action state, the offset filter decreasing an offset amount with time elapse, the first action state being a state where the total load value tends to increase and is equal to or larger than a preset determination value.

11. A non-transitory storage medium storing a program that causes a computer to execute a detection method comprising:
a step of acquiring measurement information from a load distribution sensor, the load distribution sensor detecting a distribution of a load that is received from a sole of a subject;
a step of calculating a total load value of a sole region based on the measurement information, the sole region corresponding to a position of a sole of one leg of the subject;
a step of determining an action state of the one leg based on the total load value; and
a step of starting to offset the total load value using an offset filter, in response to a determination that the action state is a first action state, the offset filter decreasing an offset amount with time elapse, the first action state being a state where the total load value tends to increase and is equal to or larger than a preset determination value.
